(19) United States
(12) Patent Application Publication
(10) Pub. No.: US 2012/0220455 A1
(43) Pub. Date: Aug. 30, 2012

(54) Azolylmethyloxiranes, Their Use for Controlling Phytopathogenic Fungi and Compositions Comprising Them

(75) Inventors: Jochen DIETZ, Mannheim (DE); Thomas Grote, Wachenheim (DE); Bernd Müller, Frankenthal (DE); Jan Klaas Lohmann, Ludwigshafen (DE); Jens Renner, Bad Durkheim (DE); Sarah Ulmschneider, Bad Durkheim (DE); Alice Glättli, Mannheim (DE)

(73) Assignee: BASF SE, Ludwigshafen (DE)

(21) Appl. No.: 13/470,512

(22) Filed: May 14, 2012

Related U.S. Application Data
(63) Continuation of application No. 12/306,027, filed on Dec. 22, 2008.

(30) Foreign Application Priority Data
Jun. 23, 2006 (EP) 06115936.4

(51) Int. Cl. A01N 43/653; A01P 3/00; C07D 407/06

(52) U.S. Cl. 504/100; 548/267.8; 514/383

(57) ABSTRACT

The present invention relates to azolylmethyloxiranes of the general formula I

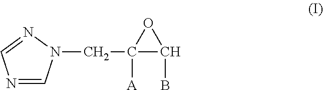

in which
A is phenyl which is substituted by an F and another substituent selected from the group consisting of Cl, C₁-C₄-alkyl, C₁-C₄-haloalkyl and C₁-C₄-alkoxy,
B is unsubstituted pyridyl, thienyl, thiazolyl, oxazolyl or furyl or is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
   with the proviso that B is not o-methylphenyl if A is 2-chloro-4-fluorophenyl,
      and to the plant-compatible acid addition salts or metal salts thereof, to the use of the compounds of the formula I for controlling phytopathogenic fungi and to compositions comprising these compounds.

This application is a continuation application of U.S. application Ser. No. 12/306,027 filed Dec. 22, 2008, the entire contents of which is hereby incorporated herein by reference. U.S. application Ser. No. 12/306,027 is a National Stage application of International Application No. PCT/EP2007/056124 filed Jun. 20, 2007, the entire contents of which is hereby incorporated herein by reference. U.S. application Ser. No. 12/306,027 also claims priority under 35 U.S.C. §119 to EP Patent Application No. 06115936.4, filed Jun. 23, 2006, the entire contents of which is hereby incorporated herein by reference.

The present invention relates to azolylmethyloxiranes of the general formula I

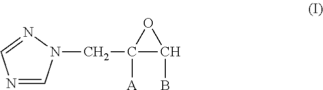

in which
A is phenyl which is substituted by an F and another substituent selected from the group consisting of Cl, C₁-C₄-alkyl, C₁-C₄-haloalkyl and C₁-C₄-alkoxy,
B is unsubstituted pyridyl, thienyl, thiazolyl, oxazolyl or furyl or is phenyl which is substituted by one to three of the following substituents: halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio or C₁-C₄-alkylthio,
   with the proviso that B is not o-methylphenyl if A is 2-chloro-4-fluorophenyl,

and to the plant-compatible acid addition salts or metal salts thereof.

Furthermore, the invention relates to the use of the compounds of the formula I for controlling phytopathogenic fungi and to compositions comprising these compounds.

Azolylmethyloxiranes, their preparation and their use in crop protection are known, for example, from EP-A 0 094 564 and EP-A 0 196 038.

Azolylmethyloxiranes which carry a hetaryl substituent on the oxirane ring are known from EP-A 0 421 125.

The azolylmethyloxiranes described already have good to very good fungicidal activity against a number of pathogens; however, it was the object of the present invention to provide novel azolylmethyloxiranes having improved fungicidal activity.

This object was achieved with the compounds of the formula I described at the outset.

Owing to the basic character of their nitrogen atoms, the compound I is capable of forming salts or adducts with inorganic or organic acids or with metal ions.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminum, tin and lead and also of transition groups one to eight, in particular chromium, manganese, iron, cobalt, nickel, copper, zinc, and others. Particular preference is given to the metal ions of the elements of transition groups of the fourth period. The metals can be present in the various valencies that they can assume.

The preparation of the compounds of the formula I is known and described in detail in EP-A 0 094 564, EP-A 0 196 038 and EP-A 0 421 125.

In the definitions of the symbols given in the formulae above, collective terms were used which are generally representative of the substituents below:

Halogen: fluorine, chlorine, bromine and iodine;

Alkyl and the alkyl moieties of composite groups such as, for example, alkylamino: saturated straight-chain or branched hydrocarbon radicals having preferably 1 to 4 carbon atoms, such as methyl, ethyl, propyl, 1-methylethyl, butyl, 1-methylpropyl, 2-methylpropyl and 1,1-dimethylethyl.

Haloalkyl: alkyl as mentioned above, where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₄)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

Alkoxy: an alkyl group as defined above which is attached via an oxygen, preferably having 1 to 4 carbon atoms. Examples of preferred alkoxy groups are: methoxy, ethoxy, n-propoxy, 1-methylethoxy, butoxy, 1-methylpropoxy, 2-methylpropoxy or 1,1-dimethylethoxy.

Haloalkoxy: alkoxy as defined above, where some or all of the hydrogen atoms in these groups are replaced by halogen atoms as described above under haloalkyl, in particular fluorine, chlorine or bromine. Examples of preferred haloalkoxy radicals are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂-C₂F₅, OCF₂-C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

Alkylthio: alkyl as defined above which is attached via a sulfur atom.

The novel compounds of the formula I comprise chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions. Preference is given here to enantiomer pairs or enantiomers with cis arrangement of ring B and triazolylmethyl substituent.

The compounds according to the invention may be present in various crystal modifications which may differ in their biological activity. They are likewise provided by the present invention.

In the compounds of the formula I according to the invention or in the compounds of the formula I used according to the invention, the following meanings of the substituents, in each case on their own or in combination, are particularly preferred. Here, the preferred substituents or preferred combinations of substituents apply, if appropriate, correspondingly to the precursors of the compounds according to the invention.

The substituent A is phenyl which is substituted by an F and a further substituent selected from the group consisting of Cl, C₁-C₄-alkyl, C₁-C₄-haloalkyl and C₁-C₄-alkoxy. According to one embodiment, one of the substituents is located in the 4-position of the phenyl ring.

According to a further embodiment, the phenyl ring is substituted in the 2,4-position.

According to a further embodiment, A is phenyl which is substituted by an F and a further substituent selected from the group consisting of Cl, methyl, trifluoromethyl and methoxy.

In a preferred embodiment, the substituent A is phenyl which is substituted by an F and a further substituent selected from the group consisting of methyl, methoxy and chlorine, such that in total 12 substituents A1 to A12 of the following formulae result:

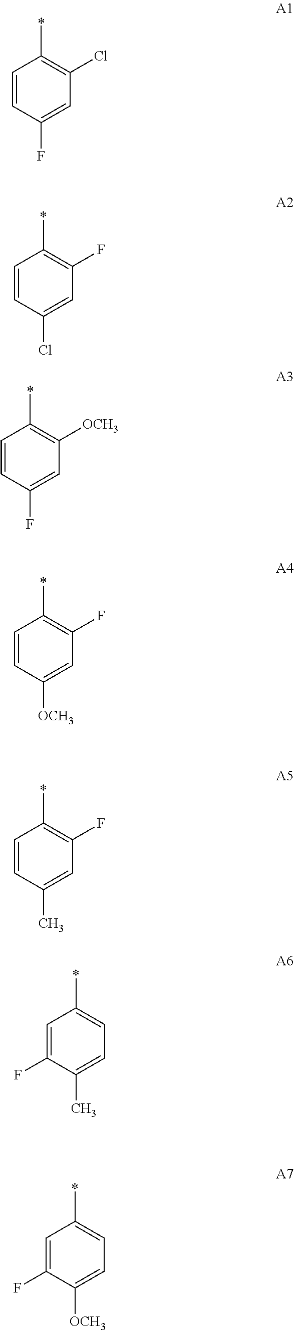

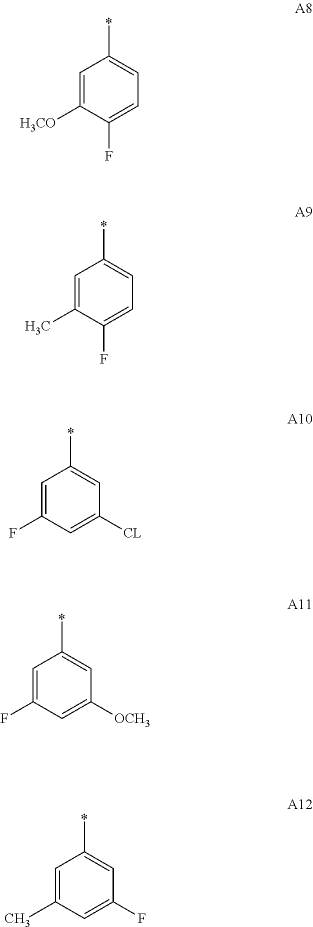

According to one embodiment, A is A1 to A5.

According to a further embodiment, A is A1, A2 or A3.

According to a preferred embodiment, A is A1 or A2.

According to a further embodiment, A is A1.

According to a further embodiment, A is A2.

According to a further embodiment, A is A3.

According to a further embodiment, A is A4.

According to a further embodiment, A is A5.

According to a further embodiment, A is A6, A7, A8 or A9.

According to a further embodiment, A is A6, A8 or A9.

According to a preferred embodiment, A is A8 or A9.

According to a particularly preferred embodiment, A is A9.

According to a further embodiment, A is A6.

According to a further embodiment, A is A7.

According to a further embodiment, A is A8.

According to a further embodiment, A is A10, A11 or A12.

According to a particularly preferred embodiment, A is A10.

According to a further embodiment, A is A11.

According to a further embodiment, A is A12.

A further embodiment relates to compounds I in which B is unsubstituted pyridyl, thienyl, thiazolyl, oxazolyl or furyl.

According to a preferred embodiment, B is pyridyl or thienyl.

According to a further preferred embodiment, B is pyridyl.

In a further embodiment of the present invention, B is phenyl which is substituted by one to three of the following substituents: halogen, NO₂, amino, C₁-C₄-alkyl, C₁-C₄-alkoxy, C₁C₄-haloalkyl, C₁-C₄-haloalkoxy, C₁-C₄alkylamino, C₁-C₄-dialkylamino, thio or C₁-C₄alkylthio.

In a further embodiment, B is phenyl which is substituted by one to three of the following substituents: halogen, C₁-C₄-alkyl, C₁-C₄alkoxy, C₁-C₄-haloalkyl or C₁-C₄-haloalkoxy.

In a preferred embodiment, B is phenyl which is substituted by one to three halogens.

In particular with a view to their use, preference is given to the compounds I according to the invention compiled in Tables 2 to 13 below. The groups mentioned for a substituent in the tables are furthermore per se, independently of the combination in which they are mentioned, a particularly preferred embodiment of the substituent in question.

TABLE 1

| Row | Substituent B |
|---|---|
| 1-1 | 2-methylphenyl |
| 1-2 | 3-methylphenyl |
| 1-3 | 4-methylphenyl |
| 1-4 | 2-methoxyphenyl |
| 1-5 | 3-methoxyphenyl |
| 1-6 | 4-methoxyphenyl |
| 1-7 | 2-chlorophenyl |
| 1-8 | 3-chlorophenyl |
| 1-9 | 4-chlorophenyl |
| 1-10 | 2-fluorophenyl |
| 1-11 | 3-fluorophenyl |
| 1-12 | 4-fluorophenyl |
| 1-13 | 2-chloro-3-methoxyphenyl |
| 1-14 | 2-chloro-4-methoxyphenyl |
| 1-15 | 2,3-dichlorophenyl |
| 1-16 | 2,4-dichlorophenyl |
| 1-17 | 3,4-dichlorophenyl |
| 1-18 | 2,3-difluorophenyl |
| 1-19 | 2,4-difluorophenyl |
| 1-20 | 2-chloro-3-fluorophenyl |
| 1-21 | 2-chloro-4-fluorophenyl |
| 1-22 | 2-pyridyl |
| 1-23 | 3-pyridyl |
| 1-24 | 4-pyridyl |
| 1-25 | 2-thienyl |
| 1-26 | 3-thienyl |
| 1-27 | thiazol-4-yl |
| 1-28 | thiazol-5-yl |
| 1-29 | oxazol-4-yl |
| 1-30 | oxazol-5-yl |
| 1-31 | 2-furyl |

Table 2

Compounds of the formula I in which A is A1 and B corresponds in each case to a substituent of a row of Table 1, with the exception of 2-methylphenyl.

Table 3

Compounds of the formula I in which A is A2 and B corresponds in each case to a substituent of a row of Table 1.

Table 4

Compounds of the formula I in which A is A3 and B corresponds in each case to a substituent of a row of Table 1.

Table 5

Compounds of the formula I in which A is A4 and B corresponds in each case to a substituent of a row of Table 1.

Table 6

Compounds of the formula I in which A is A5 and B corresponds in each case to a substituent of a row of Table 1.

Table 7

Compounds of the formula I in which A is A6 and B corresponds in each case to a substituent of a row of Table 1.

Table 8

Compounds of the formula I in which A is A7 and B corresponds in each case to a substituent of a row of Table 1.

Table 9

Compounds of the formula I in which A is A8 and B corresponds in each case to a substituent of a row of Table 1.

Table 10

Compounds of the formula I in which A is A9 and B corresponds in each case to a substituent of a row of Table 1.

Table 11

Compounds of the formula I in which A is A10 and B corresponds in each case to a substituent of a row of Table 1.

Table 12

Compounds of the formula I in which A is A11 and B corresponds in each case to a substituent of a row of Table 1.

Table 13

Compounds of the formula I in which A is A12 and B corresponds in each case to a substituent of a row of Table 1.

The compounds I are suitable as fungicides. They are distinguished by an excellent activity against a broad spectrum of phytopathogenic fungi from the class of the Ascomycetes, Deuteromycetes, Oomycetes and Basidiomycetes, in particular from the class of the Oomycetes. Some of them are systemically effective and can be used in crop protection as foliar fungicides, as fungicides for seed dressing and as soil fungicides.

They are particularly important in the control of a multitude of fungi on various crop plants, such as wheat, rye, barley, oats, rice, corn, grass, bananas, cotton, soya, coffee, sugar cane, vines, fruit and ornamental plants, and vegetable plants, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumber plants;
Fusarium and Verticillium species on various plants, such as, for example,
F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onion;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apple;
Pseudocercosporella herpotrichoides on cereals;
Pseudoperonospora on various plants, such as, for example, P. cubensis on cucumber or P. humili on hops;
Puccinia species on various plants, such as, for example, P. triticina,
P. striformins, P. hordei or P. graminis on cereals or P. asparagi on asparagus;
Pyricularia oryzae, Corticium sasakii, Sarocladium oryzae, S.attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apple.

They are particularly suitable for controlling harmful fungi from the class of the Peronosporomycetes (syn. Oomycetes), such as Peronospora species, Phytophthora species, Plasmopara viticola, Pseudoperonospora species and Pythium species.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seed or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 7Q parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.
2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers. When mixing the compounds I or the compositions comprising them with one or more further active compounds, in particular fungicides, it is in many cases possible, for example, to widen the activity spectrum or to prevent the development of resistance. In many cases, synergistic effects are obtained.

The present invention furthermore provides a combination of at least one azolylmethyloxirane of the formula I, in particular an azolylmethyloxirane disclosed in the present description as being preferred, and/or an agriculturally acceptable salt thereof and at least one further fungicidal, insecticidal, herbicidal and/or growth-regulating active compound, it being possible for a synergistic effect to occur.

The present invention also provides a pesticidal composition which comprises at least one compound of the formula I, in particular a compound of the formula I described in the present description as being preferred, and/or an agriculturally acceptable acid addition salt or metal salt thereof and at least one solid or liquid carrier. Such a pesticidal composition may comprise at least one further fungicidally, insecticidally and/or herbicidally active compound, it also being possible for a synergistic effect to occur.

The following list L of fungicides with which the compounds according to the invention can be applied together is meant to illustrate the possible combinations, but not to limit them:

List L:

Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

L-2

Carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

Azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

Nitrogenous Heterocyclyl Compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N, N-dimethyl-3-(3-bromo-6-fluoro-2-methylindole-1-sulfonyl)-[1,2,4]triazole-1-sulfonamide;

Carbamates and Dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

Other Fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, toiclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

### 1) Synthesis of 1-chloro-2-(4-chloro-2-fluorophenyl)-3-(2-chlorophenyl)propan-2-ol

Magnesium turnings (430 mg, 17.9 mmol) and iodine were added to a solution of 2-chlorobenzyl chloride (200 μl, 1.7 mmol) in anhydrous diethyl ether (20 ml). The reaction mixture was warmed slowly until the iodine color disappeared and the start of the reaction was indicated by the refluxing solvent. The remaining 2-chlorobenzyl chloride (2.0 ml, 15.3 mmol) was added dropwise such that the reaction mixture was kept at reflux. After the addition had ended, the mixture was stirred at room temperature for another 2 h and then cooled to 0° C. At this temperature, 4-chloro-2-fluorophenacetyl chloride (3.00 g, 14.5 mmol) in anhydrous toluene (10 ml) was added dropwise. The mixture was then warmed to room temperature and stirred for another 3 h. After this time, the mixture was again cooled to 0° C., and a saturated aqueous ammonium chloride solution (10 ml) was added. The organic phase was removed and the aqueous phase was extracted with ethyl acetate (2×20 ml). The combined organic extracts were dried over sodium sulfate and freed from the solvent. The crude product obtained in this manner (5.0 g) was used without purification for the next reaction step.

### 2) Synthesis of (Z)-4-chloro-1-[3-chloro-1-(2-chlorophenyl)prop-1-en-2-yl]1-2-fluorobenzene

At 0° C., first acetic anhydride (1.8 ml, 19.1 mmol) and then concentrated sulfuric acid (100 μl, 1.96 mmol) were added to a solution of 1-chloro-2-(4-chloro-2-fluorophenyl)-3-(2-chlorophenyl)propan-2-ol (5.0 g, approximately 14.5 mmol) in a 1,4-dioxane/THF mixture (44 ml, 10:1). The reaction mixture was then warmed to room temperature, stirred for another 18 h and subsequently cooled again to 0° C. At this temperature, saturated sodium chloride solution (20 ml) was added, and the mixture was neutralized using aqueous sodium hydroxide solution (6.2 ml, 50% w/w). The resulting mixture was extracted with ethyl acetate (3×20 ml), the organic phases were combined and dried over sodium sulfate. After filtration and removal of the solvent under reduced pressure, the residue was purified by column chromatography (silica gel, hexane). The appropriate fractions were combined, giving (Z)-4-chloro-1-[3-chloro-1-(2-chloro-phenyl)prop-1-en-2-yl]-2-fluorobenzene in the form of a colorless oil (1.40 g, 31% over 2 steps).

¹H NMR (300 MHz, CDCl₃) δ 8.01 (d, J=6.9 Hz, 1H), 7.46-7.37 (m, 4H), 7.22-7.10 (m, 2H), 6.8 (s, 1H), 4.50 (s, 2H).

### 3) Preparation of anti-2-(4-chloro-2-fluorophenyl)-2-(chloromethyl)-3-(2-chlorophenyl)-oxirane

Maleic anhydride (2.48 g, 25.3 mmol) and aqueous hydrogen peroxide solution (1.46 ml of a 50% strength solution, 25.3 mmol) were added to a solution of (Z)-4-chloro-1-[3-chloro-1-(2-chlorophenyl)prop-1-en-2-yl]-2-fluorobenzene (800 mg, 2.53 mmol) in acetic acid (20 ml). The reaction mixture was stirred at 45° C. for 3 d and then cooled to room temperature, and water (20 ml) and aqueous thiosulfate solution (10% solution, 4 ml) were added. The aqueous phase was extracted with dichloromethane (3×15 ml), and the combined organic phases were washed with sodium chloride solution (2×10 ml). The organic phase was dried over sodium sulfate and filtered off, and the solvent was distilled off. The residue obtained in this manner was purified by column chromatography (silica gel, 25:1 hexane/ethyl acetate). The appropriate fractions were combined, giving anti-2-(4-chloro-2-fluorophenyl)-2-(chloromethyl)-3-(2-chlorophenyl)oxirane (580 mg, 70%) in the form of a colorless solid.

¹H NMR (300 MHz, CDCl₃) δ 7.50 (t, J=9.0 Hz, 1H), 7.49-7.41 (m, 2H), 7.36-7.32 (m, 2H), 7.24-7.14 (m, 2H), 4.30 (s, 1H), 3.87 (d, J=12.0 Hz, 1H), 3.37 (d, J=12.0 Hz, 1 H).

### 4) Preparation of 1-[(anti-2-(4-chloro-2-fluorophenyl)-3-(2-chlorophenyl)oxiran-2-yl)-methyl]-1H-1,2,4-triazole

At room temperature, 1,2,4-triazole (362 mg, 5.24 mmol) and sodium hydride (130 mg, 5.42 mmol) were added to a solution of anti-2-(4-chloro-2-fluorophenyl)-2-(chloro-methyl)-3-(2-chlorophenyl)oxirane (580 mg, 1.74 mmol) in anhydrous N,N-dimethyl-formamide (20 ml). The mixture was stirred initially for 20 h at 60° C. and then for a further 4 h at 75° C. The mixture was then cooled to room temperature, diluted with ethyl acetate (20 ml) and washed with sodium chloride solution (3×15 ml). The organic phase was removed, dried over sodium sulfate and filtered, and the solvent was distilled off. The residue obtained in this manner was purified by column chromatography (silica gel, 7:3 hexane/ethyl acetate). The appropriate fractions were combined, giving the target compound (383 mg, 60%) in the form of a colorless solid.

¹H NMR (300 MHz, CDCl₃) δ 7.86 (s, 1H), 7.78 (s, 1H), 7.61-7.58 (m, 1H), 7.57-7.37 (m, 3H), 7.23-7.06 (m, 3H), 4.76 (d, J=12.0 Hz, 1H), 4.28 (s, 1H), 4.02 (d, J=12.0 Hz, 1H).

In accordance with this preparative prescription, the following compounds of the general formula 1 were prepared as racemates having a cis arrangement of triazolyl-methyl substituent and ring B of table 14:

TABLE 14

| | | | Physical data |
|---|---|---|---|
| Comp. | | | FP [° C.], ¹H-NMR |
| No. | A | B | (300 MHz, CDCl3) |
| 14.1 | 3-fluoro-4-methoxyphenyl | 2-chlorophenyl | 165-168 |
| 14.2 | 2-chloro-4-fluorophenyl | 2-fluorophenyl | 108-111 |
| 14.3 | 4-chloro-2-fluorophenyl | 4-chlorophenyl | 114-116 |
| 14.4 | 2-chloro-4-fluorophenyl | 4-fluorophenyl | 7.87 (1H), 7.82 (1H), |
| | | | 7.58 (2H), 7.18 (3H), |
| | | | 7.08 (1H), 6.90 (1H), |
| | | | 4.70 (1H), 4.22 (1H), |
| | | | 4.03(1H) |
| 14.5 | 2-chloro-4-fluorophenyl | 2-chlorophenyl | 92-97 |
| 14.6 | 4-chloro-2-fluorophenyl | 4-methylphenyl | 91-93 |
| 14.7 | 4-chloro-2-fluorophenyl | 2-chlorophenyl | 121-123 |
| 14.8 | 2-fluoro-4-chlorophenyl | 3-chlorophenyl | 7.89 (1H), 7.81 (1H), |
| | | | 7.53 (1H), 7.42 (3H), |
| | | | 7.12 (3H), 4.65 (1H), |
| | | | 4.20 (1H), 4.07 (1H) |
| 14.9 | 2-fluoro-4-chlorophenyl | 2-fluorophenyl | 150-152 |
| 14.10 | 2-fluoro-4-methoxyphenyl | 2-chlorophenyl | 115-116 |
| 14.11 | 2-fluoro-4-methoxyphenyl | 2-methylphenyl | 107-108 |
| 14.12 | 4-fluoro-2-methoxyphenyl | 2-chlorophenyl | 112-115 |
| 14.13 | 4-fluoro-2-methoxyphenyl | 2-fluorophenyl | 94-95 |
| 14.14 | 4-fluoro-2-methoxyphenyl | 2-methylphenyl | 104-106 |

Biological Tests

Greenhouse

Preparation of Active Compound

The active compounds were prepared separately or jointly as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below.

### Use Example 1

### Efficacy Against Early Blight of Tomato Caused by Alternaria solani (Alteso P1)

Leaves of potted tomato plants were sprayed to runoff point with an aqueous suspension having the active compound concentration stated below. The next day, the leaves were inoculated with an aqueous spore suspension of Alternaria solani in a 2% biomalt solution. The plants were then placed in a water vapor-saturated chamber at temperatures between 20 and 22° C. After 5 days, the disease on the untreated but infected control plants had developed to such an extent that the infection could be determined visually in %.

The plants which had been treated with an aqueous active compound preparation comprising 63 ppm of the active compounds 14.10, 14.12, 14.13 and 14.14 of table 14 showed an infection of at most 10%, whereas the untreated plants were 90% infected.

Microtest

The active compounds were formulated separately as a stock solution having a concentration of 10 000 ppm in DMSO.

### Use Example No. 2

### Activity Against Botrytis cinerea Gray Mold Pathogen in the Microtiter Test (Botrci)

The stock solution is pipetted onto a microtiter plate (MTP) and diluted to the stated active compound concentration using a malt-based aqueous nutrient medium for fungi. An aqueous spore suspension of Botrytis cinerea was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

The pathogens treated with an aqueous active compound preparation comprising 125 ppm of the active compounds 14.2, 14.3, 14.4, 14.5, 14.6, 14.7, 14.8, 14.9 and 14.11 of table 14 showed a growth of not more than 2%.

### Use Example No. 3

### Activity Against the Septoria Leaf Blotch Pathogen Septoria tritici in the Microtiter Test (Septtr)

The stock solution is pipetted onto a microtiter plate (MTP) and diluted to the stated active compound concentration using a malt-based aqueous nutrient medium for fungi. An aqueous spore suspension of Septoria tritici was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

The pathogens treated with an aqueous active compound preparation comprising 125 ppm of the active compounds 14.2, 14.3, 14.4, 14.5, 14.6, 14.7, 14.8, 14.9 and 14.11 of table 14 showed a growth of not more than 9%.

Comparative Experiment

Greenhouse

Preparation of Active Compound

The active compounds were prepared separately or jointly as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below.

### Comparative Example 1

### Efficacy Against Early Blight of Tomato Caused by Alternaria solani (Alteso P1)

Leaves of potted tomato plants were sprayed to runoff point with an aqueous suspension having the active compound concentration stated below. The next day, the leaves were inoculated with an aqueous spore suspension of Alternaria solani in a 2% biomalt solution. The plants were then placed in a water vapor-saturated chamber at temperatures between 20 and 22° C. After 5 days, the disease on the untreated but infected control plants had developed to such an extent that the infection could be determined visually in %.

TABLE 15

| | | Infection in % at 63 ppm for |
|---|---|---|
| Comp. No.: | Structure | Alteso P1 |
| 14.10 | 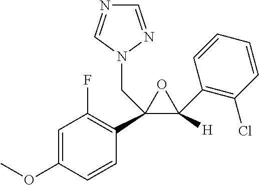 | 5 |
| As described in EP-A 0 196 038 | 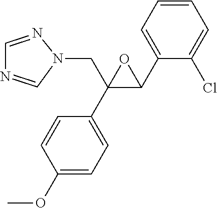 | 90 |

The biological results of table 15 show a distinctly superior fungicidal effect for inventive compound No. 14.10 compared with the structurally most similar compound of the prior art as described in EP-A 0 196 038

1. An azolylmethyloxirane of the general formula I 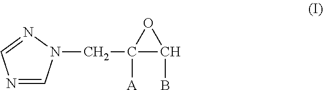
wherein
A is phenyl which is substituted by an F and another substituent selected from the group consisting of Cl, C₁-C₄-alkyl, C₁-C₄-haloalkyl and C₁-C₄-alkoxy, wherein phenyl is substituted at positions 2 and 4,
B is phenyl which is substituted by one to three halogen
or a plant-compatible acid addition salt or metal salt thereof.

2. The compound of claim 1 wherein A is 2-chloro-4-fluorophenyl, 2-fluoro-4-chlorophenyl, 2-methoxy-4-fluorophenyl, 2-fluoro-4-methoxyphenyl or 2-fluoro-4-methylphenyl.

3. A crop protection composition comprising an agriculturally acceptable solvent, auxiliary, carrier, surfactant, emulsion, paste or oil dispersion and the compound of claim 1 and/or an acid addition salt or metal salt thereof.

4. The crop protection composition of claim 3 wherein A is 2-chloro-4-fluorophenyl, 2-fluoro-4-chlorophenyl, 2-methoxy-4-fluorophenyl, 2-fluoro-4-methoxyphenyl or 2-fluoro-4-methylphenyl.

5. A seed treated with the compound of claim 1 and/or an acid addition salt or metal salt thereof.

6. The seed of claim 5 wherein A is 2-chloro-4-fluorophenyl, 2-fluoro-4-chlorophenyl, 2-methoxy-4-fluorophenyl, 2-fluoro-4-methoxyphenyl or 2-fluoro-4-methylphenyl.

7. A method for controlling phytopathogenic fungi wherein the fungi or the materials, plants, the soil or seed to be protected against fungal attack are/is treated with an effective amount of the compound of claim 1 or an acid addition salt or metal salt thereof.

8. The method of claim 7 wherein A is 2-chloro-4-fluorophenyl, 2-fluoro-4-chlorophenyl, 2-methoxy-4-fluorophenyl, 2-fluoro-4-methoxyphenyl or 2-fluoro-4-methylphenyl.
